(19) United States
(12) Patent Application Publication
(10) Pub. No.: US 2020/0383554 A1
(43) Pub. Date: Dec. 10, 2020

(54) DEPLOYABLE BALLOON ILLUMINATION FOR ENDOSCOPY

(71) Applicant: VIVID MEDICAL, INC., Palo Alto, CA (US)

(72) Inventors: Mina FARR, Palo Alto, CA (US); Stefan EBERLE, Mountain View, CA (US)

(73) Assignee: VIVID MEDICAL, INC., Palo Alto, CA (US)

(21) Appl. No.: 16/896,098

(22) Filed: Jun. 8, 2020

Related U.S. Application Data
(60) Provisional application No. 62/858,909, filed on Jun. 7, 2019.

(51) Int. Cl. A61B 1/00; A61B 1/05; A61B 1/06; A61B 1/005

(52) U.S. Cl. CPC A61B 1/00082; A61B 1/00068; A61B 1/005; A61B 1/0684; A61B 1/05

(57) ABSTRACT

A selectively deployable balloon member for insertion into and illumination of a body cavity. The selectively deployable balloon member includes a flexible tubular body having a non-deployed configuration during insertion into the body cavity and a deployed configuration after insertion into the body cavity, the flexible tubular balloon member being inflated after the insertion into the body cavity so as to be in the deployed configuration and having a reflective portion configured to reflect illuminated light received from at at least one light source disposed on an interior of the flexible tubular body when in the deployed configuration, and a transparent portion located at a distal end thereof which enables illuminated light from the light source to pass therethrough.

### CROSS-REFERENCE TO RELATED APPLICATIONS

This application claims priority U.S. Provisional Application No. 62/858,909, filed on Jun. 7, 2019. The foregoing patent application is herein incorporated by reference.

### BACKGROUND OF THE INVENTION

### 1. The Field of the Invention

The present invention relates generally to apparatus for the illumination of endoscopic and borescopic fields, in minimally invasive surgical (MIS) procedures, general or diagnostic medical or industrial procedures using endoscopes or borescopes, respectively. More particularly, embodiments of the invention relate to use of a deployable light reflective balloon for use in association with a Light Emitting Photodiode or other solid state light sources in endoscopic and borescopic procedures, as a means of assisting in illumination.

### 2. The Relevant Technology

Endoscopy is used in both diagnostic and surgical procedures. Currently, MIS procedures, as opposed to open surgical procedures, are routinely done in almost all hospitals. MIS techniques minimize trauma to the patient by eliminating the need to make large incisions. This both reduces the risk of infection and reduces the patient's hospital stay. Endoscopic procedures in MIS use different types of endoscopes as imaging means, giving the surgeon an inside-the-body view of the surgical site. Specialized endoscopes are named depending on where they are intended to look. Examples of specialized endoscopes include: cystoscopes (bladder), nephroscopes (kidney), bronchoscopes (bronchi), laryngoscopes (larynx+the voice box), otoscopes (ear), arthroscopes (joint), laparoscopes (abdomen), gastrointestinal endoscopes, and specialized stereo endoscopes used as laparoscopes or for endoscopic surgery.

Such endoscopes may be inserted, for example, through a tiny surgical incision to view joints or organs in the chest or abdominal cavity. More often, an endoscope is inserted into a natural body orifice such as the nose, mouth, anus, bladder, or vagina. There are three basic types of endoscopes: rigid, semi-rigid, and flexible. The rigid endoscope comes in a variety of diameters, lengths, and various angles of view, such as zero, 30 or 70 deg. endoscopes and used depending on the requirements of the procedure. Typical endoscopic procedures require a large amount of equipment. The main equipment used in conjunction with the visual part of the endoscopic surgery are the endoscope body, fiber optics illumination bundles, illumination light source, light source controller, imaging camera, camera control module, and video display unit.

It can be advantageous to reduce the number of incisions as well as the size of the incision as much as possible in an endoscopic surgery. Normally a separate port is necessary to be used with a large diameter endoscope that takes the entire opening of the port, cannula or catheter once access to inside the body is obtained. Space is also very limited at the proximal end of the port and tools and endoscopes with proximal camera, are bulky and heavy, often propped up and locked in position with secondary mechanism that may physically interfere with other devices used by the surgeon, especially if multiple ports are close to one another, or in Single Port Procedures.

Another common problem that occurs with endoscopic procedures is that, because the endoscope is inserted into the body, the cavity being imaged by the endoscope is small and difficult to view. One way to obtain better images is to insufflate the cavity with gas to increase the volume of the area being imaged. Insufflation can be problematic because of inadequate seals between the port opening and the endoscopic device used. In addition, the smallness of the space may cause too much contact with the endoscope, which may result in the endoscope becoming smeared with blood and liquids that obscure the view for the camera on the endoscope to capture images of the cavity. In which case the procedure has to be stopped, the endoscope taken out, wiped clean and put back into the port to resume the procedure.

The quality of images obtained by an endoscope is also partly dependent on the quality of illumination, and not just resolving power of the imaging lenses and sensor sampling frequency. Because of the limited space at the distal end of the endoscope, there is limited room to place a fiber optic or LED ring illuminator around the front aperture of imaging optics of the endoscope. In addition due to limitations in thermal heat management, limited and very low power solid state illumination sources can be placed at the distal tip of the endoscope where there is limited space for heat from the LED sources, for example, to be successfully kept away from human tissue. Consequently, heat emitted from the light sources may come in contact with human tissue during the procedure. Silicone dome lenses are commonly used on LED light sources to improve light extraction and efficacy of the light source. One disadvantage of such configurations, however, is that these dome-over-molds also increase the size of the LED light sources since there is limited or no space at the distal tip of an endoscope to also use illumination optics in conjunction with the solid state light sources which control the illumination profile over the object/tissue within the Field of View (FOV) of the imaging optics.

### BRIEF SUMMARY OF THE INVENTION

These and other limitations may be overcome by embodiments of the invention which relate to a deployable balloon illumination system that can be used in minimally invasive surgical procedures and/or diagnostic procedures in order to assist in illuminating the cavity. The deployable balloon illumination system not only provides illumination optics conditioning the illumination profile to best fit the imaging optics FOV, it also increases light extraction from the LEDs more efficiently so higher light efficacy can be achieved. Deployable balloon illumination system, with an inflatable balloon or balloons allows positioning of the solid state illumination light sources away from the very front surface of the endoscope where a ring illumination would need to be otherwise disposed, allowing the entire cross section of the endoscope distal tip to be used for larger imaging optics, thus enabling a higher numerical aperture imaging optics to be employed at the distal tip, which enables better light collection for imaging and provides higher resolution in the endoscope. Or the saved space where a ring illumination would need to be disposed otherwise, can serve as irrigation, suction or working catheter interior to the deployable balloon illuminator within the endoscope profile. The inflated balloon can also act not only as heat dissipation reservoir or heat sink with much larger heat dissipation surface for the solid state light sources, allowing higher power solid state light sources to be used and run at higher currents, but also acts as a soft and deformable safety buffer for any tissue that could come in contact with the distal tip of the endoscope, as well as keeping the imaging optics aperture away from any liquid or blood that could smear onto the imaging lenses which would otherwise require cleaning mid-procedure. Within the internal lumens of the body, the inflatable illumination balloon can also be used to keep away or plug the internal lumen in the body to allow effective local inflation of he body lumen with air, to visualize and operate on, or with selective inflation and deflation of multiple illumination balloons used as means for steering a flexible endoscope or catheter inside a internal lumen or body cavity under endoscopic procedure. According to some embodiments, the deployable balloon illumination system may be implemented in parts (multiple inflatable sections), have a reduced, or a very small and flat, shape when in a non-deployed state so as to be electively located inside a surgical port or cannula for articulation and deployment. In some of the embodiments, the deployable balloon illumination system includes a reflective and a transmissive portion, with specific optical characteristics to, reflect, transmit or with features to scatter light, so as to assist in effective illumination of the body cavity under observation.

In some embodiments, a selectively deployable balloon member for insertion into and illumination of a body cavity is described. The selectively inflatable deployable balloon member includes a flexible tubular body having a non-deployed configuration during insertion into the body cavity and a deployed configuration after insertion into the body cavity. A very thin portion of the flexible tubular body is configured to be selectively inflated after the insertion into the body cavity so as to be in the deployed configuration and having a reflective portion configured to reflect illuminated light received from at least one light source disposed on an interior surface of the flexible tubular body when in the deployed configuration. A transparent portion is built into the flexible tubular body over the light sources being or so as to come into intimate contact with the illumination sources located on the inner surface of the tubular body as the balloon is inflated. The transparent portion enables effective extraction of the light from the illumination sources allowing the illuminated light from the light source to pass therethrough the inflated balloon.

In some embodiments, the flexible tubular body may be configured to be filled with gel or fluid, such as saline, when in the deployed position. The gel or fluid may have a higher index of refraction than air so as to better extract light from the illumination light sources.

In some embodiments, the flexible tubular body may have a heart, apple, or bulb shape, or an asymmetric shape when in the deployed position, that is fully or partially deployed as needed.

In some embodiments, the thin flexible membrane of the flexible tubular body is deployed outwardly or inwardly (or in both directions) via a very small diameter air, liquid, or gel feed tube which inflates one or multiple balloon illumination systems, that is built in the wall or interior of the flexible tubular body, allowing deployment from the proximal end source of air, liquid or gel, or a separate reservoir built in the device

In some embodiments, the flexible tubular body has a toroid shape with a recess formed therein which permanently or selectively houses a camera of a medical scope, or where a traditional rigid glass lens endoscope with proximal end camera can be inserted in the flexible tubular body, which itself can have a rigid portion.

In some embodiments, the at least one light source may emit light in laterally from the interior to the flexible tubular body, the light being reflected from the reflective portion of the balloon, having passed through the air/liquid/gel material that was used to inflate the balloon, toward the transparent portion of the flexible tubular body, where scattering or lensing features such as Fresnel lens, could be used or molded within or on the reflective or transparent portions of the flexible tubular body to condition the illumination light further or assist in better light extraction.

In some embodiments, the light source may be a light emitting diode or VCSEL of various wavelengths that can be turned on and off selectively based on the imaging optics used.

Another embodiment of the invention corresponds to a device for insertion into and illumination of a body cavity. The device includes a plurality of selectively deployable balloon members having a non-deployed configuration during insertion into the body cavity and a deployed configuration after insertion into the body cavity, the selectively deployable balloon members being inflated after the insertion into the body cavity so as to be in the deployed configuration and having a reflective portion configured to reflect illuminated light received from at least one light source disposed on an interior of the selectively deployable balloon member when in the deployed configuration, and a transparent portion located at a distal end thereof which enables illuminated light from the at least one light source to pass therethrough.

Another embodiment of the invention includes an illumination device for insertion into a body cavity. The device includes an elongate hollow tube with an internal lumen between a distal opening at a distal end and a proximal opening at a proximal end, the elongate hollow tube including a rigid distal portion associated with the distal opening configured to be at least partially inserted into the body cavity, a selectively deployable balloon member operably coupled to the rigid distal portion and having an insertion position and a deployed position. The a selectively deployable balloon member is operably coupled to the rigid distal portion and has an insertion position and a deployed position. The selectively deployable balloon member has an interior portion configured to receive light from at least one electro-optic illumination element coupled to the selectively deployable balloon member, wherein the at least one electro-optic illumination element provides illumination at one or more electromagnetic wavelengths and is configured to be selectively turned on or off. The selectively deployable balloon member also has a reflective portion configured to reflect illuminated light, a transparent portion located at a distal end thereof which enables illuminated light to pass therethrough. The illumination device also includes an imaging element housing portion permanently or selectively plugged into and positioned in the rigid distal portion which is configured to house an imaging element when coupled thereto. The at least one electro-optic illumination element is disposed within an interior of the selectively deployable balloon member and is configured such that when the at least one electro-optic illumination element is turned on, light from the at least one electro-optic illumination element is reflected or scattered from the reflective or scattering portion of the selectively deployable balloon member and is transmitted through the transparent portion of the selectively deployable balloon member, which may possess light scattering and wavelength as well as directional mixing properties, towards the rigid distal portion where the imaging element is disposed to exit the balloon illumination system through a transparent or scattering front surface positioned around the imaging element in the deployed position.

In some embodiments, This Summary is provided to introduce a selection of concepts in a simplified form that are further described below in the Detailed Description. The embodiments provided do not limit the disclosure but provide scenarios to aid understanding thereof. The Summary is not intended to identify key features or essential features of the claimed subject matter, nor is it intended to be used as an aid in determining the scope of the claimed subject matter.

### BRIEF DESCRIPTION OF THE DRAWINGS

To further clarify the above and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a selectively deployable balloon illuminator for insertion into and illumination of a body cavity and in association with a distal tip endoscope according to one embodiment of the invention;

FIG. 2A illustrates a side view of the deployable balloon illuminator and endoscope of FIG. 1;

FIG. 2B illustrates a top view of the deployable balloon illuminator and endoscope of FIG. 1, where the distal tip endoscope could be built in within the balloon illuminator body as an entirely single use unit, or an independently reusable encapsulated distal camera module that is plugged into to the balloon illuminator body;

FIG. 3 illustrates a cross-sectional view of the deployable balloon illuminator and endoscope of FIGS. 1 and 2A-2B, where the distal tip camera module is built-into the balloon illuminator body and the solid state illuminators instead of facing the direction of camera view are mounted orthogonally on the body of the distal tip camera module;

FIG. 4A is a cross-sectional view illustrating an alternative embodiment comprising a dual distal tip camera module endoscope, with side solid state illuminators to be used with a deployable balloon illuminator for Stereoscopic/3D viewing;

FIG. 4B is top view illustrating an alternative embodiment comprising a single elliptical deployable balloon illuminator configuration, housed over, or inserted over the 3D endoscope of FIG. 4A;

FIGS. 5A to 5C illustrate another alternative multiple camera configuration endoscope, where various wavelengths of light could be used imaging on separate image sensors within the deployable balloon illuminator;

FIGS. 6A to 6E illustrate the distal tip of a port, flexible, or articulating catheter or cannula equipped with a deployable balloon illuminator where imaging endoscope and other tools can be inserted through the port, flexible and articulating cather and illuminated with the deployable balloon illumimator:

FIGS. 7A-7B and 8A-8B illustrate another configuration of a non-symmetric deployment of the balloon illuminator for use in association with a rigid port or flexible catheter, equipped with a distal camera that is made to be side deployed by the deployment of the balloon illumination system, allowing a larger working channel inside the rigid port or flexible catheter for access inside the body;

FIGS. 9A-9D illustrate a variety of shapes of a deployable balloon illuminator which may be used in association with the invention, where the body of the deployable balloon can be also utilized as a soft flexible interface or membrane providing space or distance from internal organs or to close a distance within an internal organ though the procedure under visualization; and

FIGS. 10A-10D illustrate a deployable balloon illuminator having two separately deployable balloon portions that can be selectively deployed inside the body.

### DETAILED DESCRIPTION OF THE EXEMPLARY EMBODIMENTS

Exemplary embodiments of the invention concern a deployable balloon illuminator which may be used as a component of or in association with an endoscope, surgical port, a catheter or surgical tool body, with or without access ports, on a flexible, partially flexible, articulating or rigid device body. Embodiments may employ or be used in association with monochromatic or polychromatic, in various visible or non-visible electromagnetic wave spectrum, solid state light sources such as high power Light Emitting Devices (LEDs), Laser Diodes. Surface Emitting Vertical Cavity Lasers (VSCELs) as a means of illumination in a diagnostic or surgical endoscopic procedures, or functional borescopic systems. In particular, these solid state light sources are incorporated near or at the distal end, or otherwise on the body of the endoscope, borescope, surgical or industrial tools, and the tip end of cannulas and other functional devices. They can also be incorporated in an illumination body that is inserted separately, or in conjunction with a lighted or dark scope, into the body. The illumination of an object inside a body, a body herein being defined as at least a portion of a human, animal or physical object not easily accessible, is performed to actively modify the body or a dye used inside the body, to detect the modified light, image the object, or manipulate a change in the object. The solid state illumination schemes of the present invention can replace, or can be used in addition to, the conventional fiber optic or solid state source illumination system and other diagnostic devices such as ultrasound imaging, or functional tools such as surgical tools used in endoscopy and borescopy.

The embodiments described herein utilize deployable balloon mechanisms, which as are described more fully herein, may have a variety of different shapes and be comprised of different materials so as to have different transparencies, elasticities, or any number of other suitable and advantageous optical or non-optical mechanical and or electrical properties. As is described herein, the deployable balloon mechanisms are configured to temporarily increase the volume of the endoscope body in its illumination system to allow effective illumination of the area to be imaged, protect the imaging components, and help keep solid state illuminators away from body organs, lumen walls, fluid, blood, or other parts and tools inside the examination body, dissipate the heat from high power solid state light sources and chip on tip imaging electronics, and to improve the ability for the camera to capture images of the cavity by allowing a larger diameter conventional or chip on tip imaging endoscope to be used without a ring illuminator fiber optic or solid state light sources around the imaging endoscope distal aperture.

As may be understood, the use of solid state light sources in association with or as a single component with a deploy able balloon and an imaging system inside a cavity in the body, replaces variety of instruments and parts otherwise needed for the same purposes, such as an external light source, fiber light guides, and a means of transmitting the light to the desired object. Further, the embodiments described herein may replace the need for a ring shaped illuminator at the very distal tip surface of the device and allow entire diameter of the imaging endoscope to be used for the imaging optics, image sensor, working channel for the endoscope or catheter for introduction of other tools, and means for articulation and navigation of the endoscope, tool or catheter. Consequently, the embodiments described herein provide a compact, effective, and easy to operate endoscopic vision system that can be made as a separate reusable endoscope, plugged into or inserted into the deployable balloon illumination body (such as chip on the tip endoscope or traditional rigid glass imaging endoscope), which can be made inexpensively and which is exchangeable for variety of illumination characteristics and types as well as in variety of illumination wavelengths required for a specific procedure, and which can be made disposable after each use.

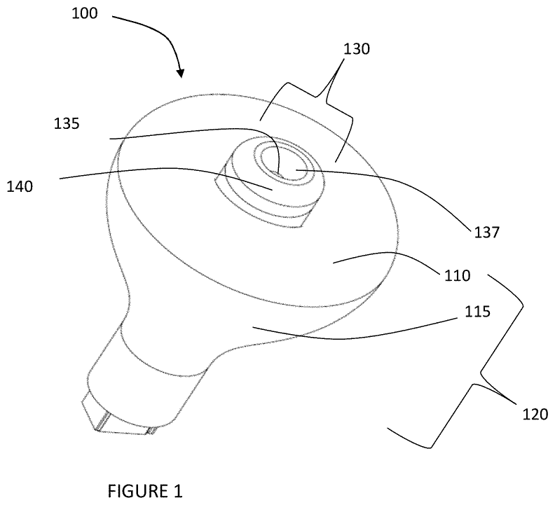

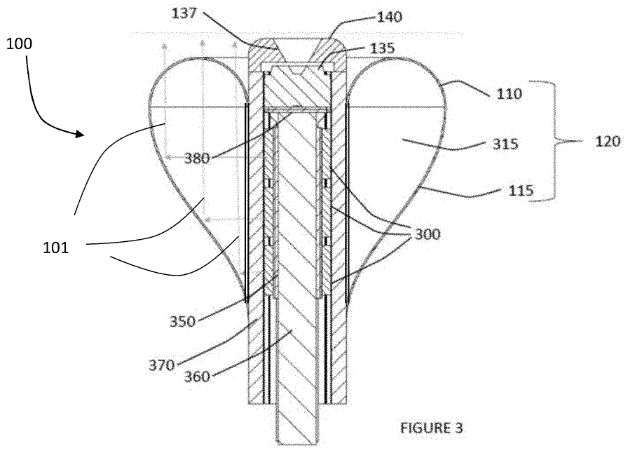

FIG. 1 illustrates an endoscope system 100 including a deployable balloon illuminator (DBI) 120 in association with various components of an endoscope, including an endoscope vision system 130 and an endoscope camera 135. The deployable balloon illuminator 120 which may be used in association with illumination sources (such the illumination sources 300 described more fully with respect to FIG. 3), that are embodied on the body of the distal tip instead of the front surface of the distal tip facing the object, close to the distal tip or conveniently located within the device body or otherwise at the distal portion of a short light guiding component that does not need to extend the entire body of the device, described herein and an imaging camera 135 that can be disposed within the DBI 120 or otherwise plugged into or inserted into the DBI 120 in order to provide an improved illumination and image capturing device as a component of an endoscope. In the configuration shown in FIG. 1, the DBI 120 is comprised as an integrated or plugged in component of in an integrated endoscope system 100 along with an endoscope vision system 130 which may include an imaging camera 135 or other optical components along with the necessary electrical, mechanical, and optical connections which may be required for such a system. It should be noted, however, that the DBI 120, may be a separate component which is used in association with other endoscopes or imaging systems currently used in the art, such as a chip on the tip flexible endoscope, or conventional glass lens rigid endoscope with or without conventional ring illumination systems of their own. Examples of such components are described more for fully below. For simplicity sake, however, various Figures described herein describe aspects of the DBI 120 as an integrated component of an endoscope system 100, noting that the LED sources, as an example of illumination sources, could be part of an inserted imaging endoscope body or permanently built into the interior wall of the DBI with electrical power and control lines provided for the LED illumination sources through the hollow flexible, rigid, or partly flexible tubular body of the DBI.

Further, in some instances, the DBI tubular body 120, or the DBI 120 with the LED illumination sources included therein may be designed to be single-use or disposable.

In the embodiment shown in FIG. 1, the DBI 120 of the endoscope 100, where the imaging portion can be made detachable, includes a transparent portion 110 towards the distal end of the endoscope system 100, which could have optical surface characteristics such as lensing or focusing effects with its special optical profile as an interface between air and the liquid or gel index of refraction that has filled the DBI 120 in its deployed configuration. This lensing or focusing effect can further be tunable for an adjustable type of illumination with the amount of inflation of the DBI 120 (inflation level, or sectional and sequential inflation) with the liquid or gel solution, where the DBI 120 take a specific shape based on the specific imaging needs of the endoscope with various DBI fill levels. The transparent portion 110 is designed to enable light from the illumination sources 300 (shown in FIG. 3) disposed on the interior of the endoscope system 100 to escape the DBI 120 and transmit there through toward an area of interest within the cavity to be illuminated. The transparent portion 110 surface could provide a smooth optical surface or light scattering roughness or surface lens such as Fresnel lens, or beads to improve extraction of the light from the DBI 120, or otherwise directing the output light based on the application. The DBI 120 also includes a reflective portion 115 proximal to the transparent portion 110, the reflective portion 115 being configured to direct light from the illumination sources 300, or otherwise direct the light disposed on the interior thereof toward the reflective portion and the distal end of the endoscope 100 so as to improve the illumination of the cavity, where the DBI 120, along with the air, liquid, or gel disposed within all operate as a part of the illumination system itself. The reflective portion 115 could also take advantage of Total Internal Reflection (TIR) of the illumination light at this surface when the DBI is inflated with higher index liquid or gel material. The reflective portion 115 could also take advantage of scattering surface that provides mixing and conditioning function for the light from multiple solid state light sources feeding light of various wavelengths feeding light into the DBI from various internal positions or otherwise deployed inside the device body.

As is further shown in FIG. 1, the endoscope system 100 also includes an endoscope vision system 130 that can be permanently attached to or temporarily plugged into the DBI 120. In the configuration shown in FIG. 1, the endoscope vision system 130 includes a camera 135 which is slightly recessed or can protrude the most distal end of the endoscope system 100 with its own separately recessed portion 137. As may be understood, this slight recess 137 of the camera 135 improves the ability of the camera 135 to capture images by further avoiding contact with fluids and/or blood within the cavity.

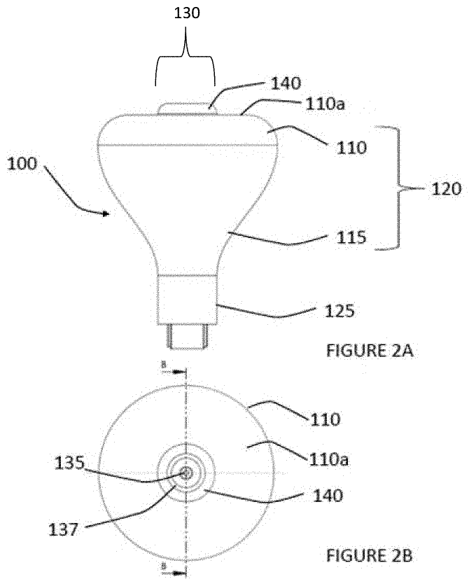

FIGS. 2A-2B illustrate side and top views of the DBI 120 and endoscope system 100 shown in FIG. 1, respectively. As is illustrated in FIG. 2A, the distal portion of the endoscope system 100 includes a protruding portion or endcap 140 which protrudes from a distal surface 110a of the transparent portion 110 of the DBI 120, providing the camera lens recess 137 from the endcap 140. Alternatively the endcap 140 which positions the imaging camera 135 at a recessed position 137, can itself be further recessed and positioned behind the DBI distal surface 110a (not shown in the Figures) to provide extra protection for the camera lens from blood and liquid splatter during the procedure or from it coming in close contact with objects or organs inside the cavity.

FIG. 3 is a cross sectional view of the endoscope system 100 and DBI 120 shown in FIGS. 1 and 2A and 2B. As is shown in this configuration, the endoscope 100 includes an endoscope tube 370 (made selectively transparent or with optical filtering properties) where a separate endoscope or imaging camera module can be plugged or inserted into the DBI 120 from the proximal end until its imaging camera optics is positioned at a recess by the endcap 140, or alternatively built into the DBI 120 as a single unit within the endoscope tube 370 as depicted in FIG. 3. The endoscope tube 370 is also used to manipulate and direct the endoscope system 100 as it is guided in to the desired position within a patient. The entire distal tip portion of endoscope system 100 could be mounted on a flexible body, that can be passively guided or articulating body, so the endoscope distal tip portion depicted in FIG. 3 can be articulated, positioned or pointed towards the area of interest. The endoscope system 100 could also include a plurality of illumination sources 300, or these solid state illumination sources could be part of the endoscope tube 370 that is the inner tube of the DBI 120. In this embodiment, the illumination sources 300 comprise a series of light emitting diodes (LEDs), although different solid state light sources or combination of these sources can be used in association with the LEDs or in place of the LEDs as illumination sources 300 to perform diagnostic, such as cancer detection, using specific wavelength fluorescence excitation sources, with specifically optically filtered imaging mechanism, as well as surgical or other functions on a body. A variety of illumination sources 300 can be incorporated within the endoscope body as in FIG. 3. For example, the illumination sources 300 may be positioned interior to an endoscope tube 370 which is made optically transparent or with transparent, or light filtering windows in front of the light sources, or the illumination source 300 can be installed within the transparent endoscope tube wall 370 itself that could have for example dichroic thin film coating on its surface, where a separate imaging endoscope can be inserted into the DBI 120). The illumination sources 300 can work in conjunction with one another and other devices to image, detect or modify the object A variety of illumination sources 300 (as part of the inserted endoscope or as part of the DBI 120 or both), can be turned on and off in specific sequences, time synchronized with the frame rate of one or multiple imagers, to detect specific sequence of images in variety of illumination wavelengths, such as RBG. RGB+other visible component wavelengths, infrared, near infrared, uv, combination or sequence thereof, using RGB and/or Black and White Imagers.

As is shown in FIG. 3, the illumination sources 300 are connected to a rigid or flexible printed circuit board (PCB) 350, which in this configuration provides both power and the ability to control the illumination sources, and are also connected to or come into intimate contact with a heat-sinking rod 360 behind the inserted or built in imaging camera 135, where independently or together with the DBI 120, the illumination sources 300 can dissipate any unwanted or unnecessary heat from the distal tip of the device and away from the patient internal organs and tissue. The illumination sources 300 are disposed so as to emit light, shown as 101 (in its most simplistic light path) in FIG. 3, laterally from the sides of the interior transparent endoscope tube 370 (or through transparent, or filtering windows within the endoscope tube 370), at a specific angle or location from the interior reflective surfaces of the reflective portion of the deployable balloon 115 towards the distal end of the DBI 120 comprising the transparent portion 110, all together comprising the Illumination System. The reflected light then illuminates the area of the cavity to as to improve and enable special imaging capabilities of the endoscope system 100, where the index of refraction of the material the DBI 120 is filled with, as well as the surface profile, micro or miniature molded plastic lenses or array of surface lenses, transparent or reflective beads, filtering, or polarizing, scattering or non scattering features of surfaces the reflective portion 115 and transparent portion 110, are designed together in the Illumination System to provide, conditioning, mixing, filtering, and pointing functions on the illumination light, and to spot light illumination profile necessary for best imaging.

In some embodiments, the DBI 120 may have an internal window at least the same size as the in front of the illumination sources 300, that if filled with an appropriately designed refractive index material to improve light extraction from the illumination sources 300 into the DBI 120. The internal tubular body of the DBI 120 or endoscope tube 370 can be made transparent in front of the illumination sources 300 and reflective otherwise to trap light inside the DBI 120, as it reflects back and forth towards the exit transparent surface 110. The DBI's 120 internal tubular surface in front of the illumination sources 300 or endoscope tube 370, can be also made very thin and flexible, so when the DBI 120 is deployed, this flexible tubular portion inside also flexibly presses against the illumination sources 300 to make an intimate contact with the illumination sources, for better light extraction from the illumination sources 30), especially when the DBI 120 is filled with higher index matching material, such as water or gel, such as the light from sources 300 travels only in higher refractive index medium until its exit from transparent surface 110.

The camera 135 is also connected to a rigid or flexible PCB 380 which controls and powers the camera 135 and it should be understood that although not expressly described herein, the endoscope system 100 also includes the necessary wiring and electrical connections necessary to power and control both the camera 135 and the illumination sources together or separately. The camera electronic connections could provide analog or digital means of imaging data transmission, in any of the variety of protocols such as MIPI, LVDS, USB/UVS, or otherwise, where the camera clock signal can be used in common with the illumination source on/off signals or with one or more other camera imagers. It should be understood that a variety of configurations may be used to provide the wiring and electrical connections for powering and controlling the camera 135, illumination sources 300 one of which is through separate USB devices controlled through a USB HUB, and the ability to selectively inflate and deflate the deployable balloon portion 120 of the endoscope system 100 through mechanical or automated inlet valves positioned inside or outside the endoscope system 100.

Examples of endoscopes which include examples of additional mechanical and electrical elements, connections, and the illuminating sources which may be used in association and in addition with this invention are found in at least U.S. Pat. Nos. 8,480,566 and 9,033,870, which are both herein incorporated by reference in their entirety.

In one embodiment, a portion of the heat generated by the illumination sources 300 is transferred to the camera 135 so as to defog the lens and maintain the quality of the images captured by the camera 135. More specifically, the heat from the illumination sources 300 may provide the proper temperature setting to avoid any condensation on an optical window or lens of the camera 135 which may accumulate during operation. Additionally, any heat from the illumination sources 300 may be used to warm the distal end of the cold endoscope 100 when it is inserted into the warm and humid body cavity. In turn a separate low power infrared LED can also be used for the purpose of heating the endoscope tip.

The DBI 120 may be inflated after the endoscope system 100 has reached its desired position within a patient or it may be entirely, partially deployed, or sectionally deployed at any desired point. The DBI 120 may be inflated by any number of known materials, including a variety of gases or liquids generally used in the medical field, such as a saline solution. The deployable balloon 120 may be deployed through a number of means, such as via a tube or other connection which inflates the interior 315 of the DBI 120, using an internal reservoir or bladder, and/or an external syringe and valves mechanism commonly used in the medical filed in inflating catheter balloons, endrotracheal intubation tube cuffs, used proximally, or any separate or device embodied prefilled reservoir in place of the air or liquid filled syringe, once the endoscope system 100 has reached a desired position or an area of a patient which is desired to be imaged and which also may require additional illumination and/or clearance around the camera 135 so as to improve the image capturing process. Subsequently, in one embodiment, after the imaging process is complete, the DBI 120 or sections of it may be deflated so as to reduce the profile of the endoscope system 10), using the same means such as a deflating syringe and proximal valve opening mechanism, and better facilitate the removal of the endoscope 100 from a patient.

As may be understood by one of skill in the art, one benefit of inflating the DBI 120 during the imaging process is that the air, liquid, or other material disposed in the interior 315 of the DBI 120 assists in isolating the patient anatomy from any heat produced by the illumination sources 300. As such, inflating the DBI 120 during guidance inside a body lumen, partially, completely or in sections, could aid the advancement and placement of a flexible or articulating endoscope through a natural orifice, where the embodiments described herein provide a safe and reliable way to capture improved endoscopic images without increasing the risk of burn or other harm to the patient during the procedure. Furthermore the inflated balloon illuminator could be used to plug an internal lumen or section of the body, where the working and imaging area can be filled with air for clear viewing within the working distance in front of the DBI.

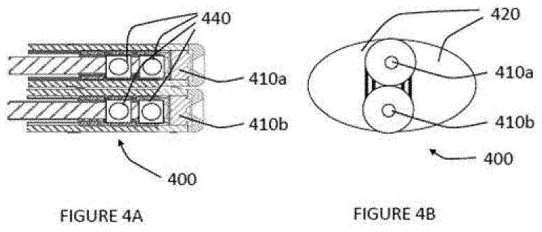

FIGS. 4A and 4B illustrate an alternative configuration wherein the endoscope 400 includes a dual imaging endoscope cameras, which may be inserted or built in within the deployable balloon illumination system or a single balloon made to inflate only in a specific desired profile, or multiple section balloons inflated appropriately. In this embodiment, the endoscope 400 includes two cameras 410a and 410b disposed in a side-by-side configuration, with a corresponding array of LEDs 440 built within the outer wall of the endoscope 400. The two cameras could detect left and right images in stereoscopic 3D imaging, and may be set to detect specific imaging content such as in visible light or near infrared for ICG or otherwise fluorescence imaging, where the two camera video signals may be clocked together, or used separately and displayed in stereoscopic 3D viewing or otherwise overlaid to show various imaging modes of the same scene, such as fluorescence and visible light images the operator. As is shown in FIG. 4B, when the dual deployable balloon sections in DBI 420 are inflated, the inflation occurs orthogonal to the axis of the two cameras 410a and 410b, which could have a non-circular deployed profile possibly opposite to the DBI cross sectional profile before deployment.

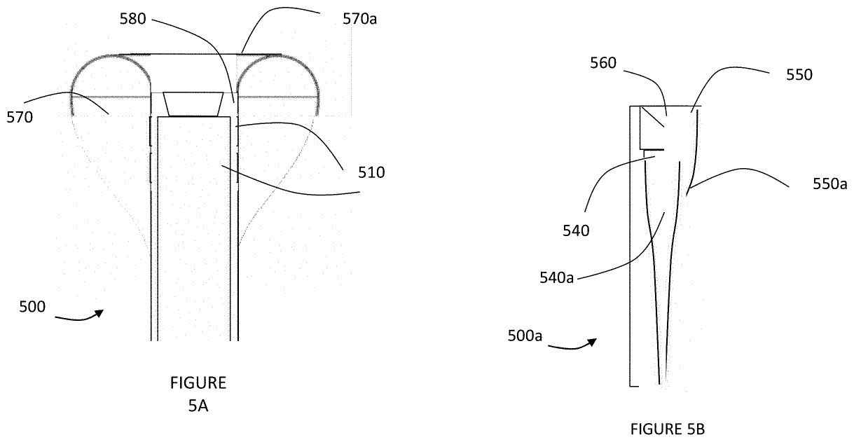

FIGS. 5A to 5C illustrate another alternative configuration of a DBI 500. In this configuration, the DBI 500 is used in independent endoscope camera system 500a, which can be independently inserted into and used in association with the DBI 500 up to and positioned by the endcap 580. Additionally. FIGS. 5A-5C illustrate an alternate dual camera imaging system, that could be used for concurrent visible and or fluorescence imaging, where the two camera imaging is systems are split using a wavelength sensitive beam splitter 56 permanently housed, or otherwise inserted within a common endoscope balloon illuminator 500. In this configuration, the illumination sources 510 built-in on the inside surface of the flexible body DBI 500 in FIG. 5A. The illumination sources 510 may comprise visible light, white, near infrared, infrared, ultraviolet LEDs, or a series of vertical-cavity surface-emitting lasers (VCSEL) matching the beam splitting thin film dichroic optical filters deposited on the various surfaces of the beam splitter 560 to direct the specific wavelengths of light to the specific image sensor at a specific time that the corresponding light source is turned on, such that the specific frames of the image sensors can capture a synchronized spectral images of the scene under observation. In this embodiment, in addition to acting as a balloon which enlarges the cavity the endoscope is inserted in (within a body lumen or otherwise), positioning of an effective and efficient illumination light with focusing and spot light properties around the endoscope imaging window in this configuration the deployable balloon 570 of the DBI 500 acts as a visible light, white, near infrared. IR, or UV light mixer and reflector which causes a uniform illumination in visible light, white, near infrared, IR, or UV LED light towards the target area of the cavity at the distal surface 570a of the DBI 500, and may turned on and off at a specific sequence, clocked with the two cameras, or both cameras and illuminators on at the same time. The beam splitter 560 and dual cameras 550 and 540 and their associated optical axes may be aligned in such manner to superimpose the two camera 550 and 540 images on the display, observing the same scene with different imaging attributes.

FIG. 5B alternatively illustrates the internal optical components of the camera system 135 with its recessed distal end imaging lens common to both camera imaging system optical path with respect to the DBI balloon 570 distal surface 570a. More particularly, the camera imaging system 135 may include a visible light RGB imager 540, flexible printed circuits (FPCs) 540a and 540b for cameras 540 and 550, respectively, along with a wavelength cut filter beam splitter 560, and a near infrared, IR sensitive black and white imager 550 providing a camera system capable of capturing both visible light and fluorescent light with a single sensor sensitive to both wavelength ranges, or using multiple internal image sensors with specific sensitivity to the range of wavelengths of light.

Although in the previous embodiments, the DBI 120 is described as a component in an integrated camera 135 as a component of an endoscope system 100, in other embodiments the DBI 120 provides an endoscope tube 370 (as shown in FIG. 3) where a conventional rigid glass endoscope or a chip on the tip rigid, flexible or articulating endoscope can be inserted into the DBI 120. The DBI may include a stopping endcap built at the distal tip of the endoscope tube 370, with a camera recess 580 configuration designed into the DBI 500. Consequently, the invention described herein is not limited to an integrated endoscope or co-positioned and articulated endoscope.

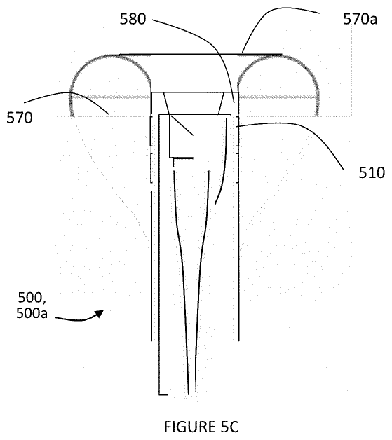

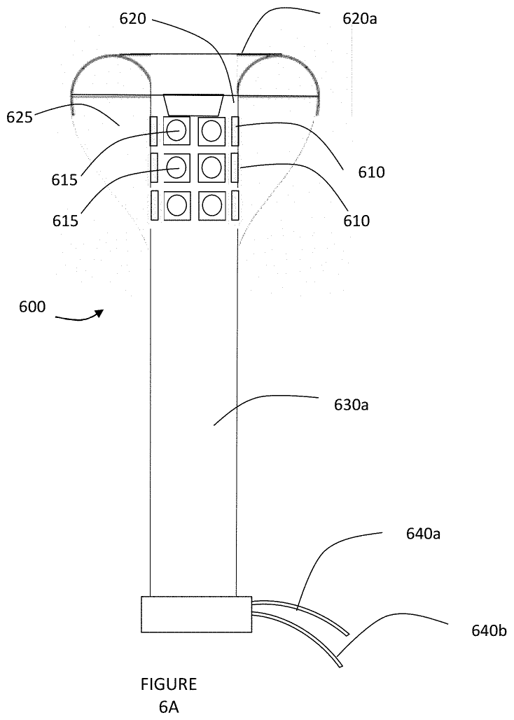

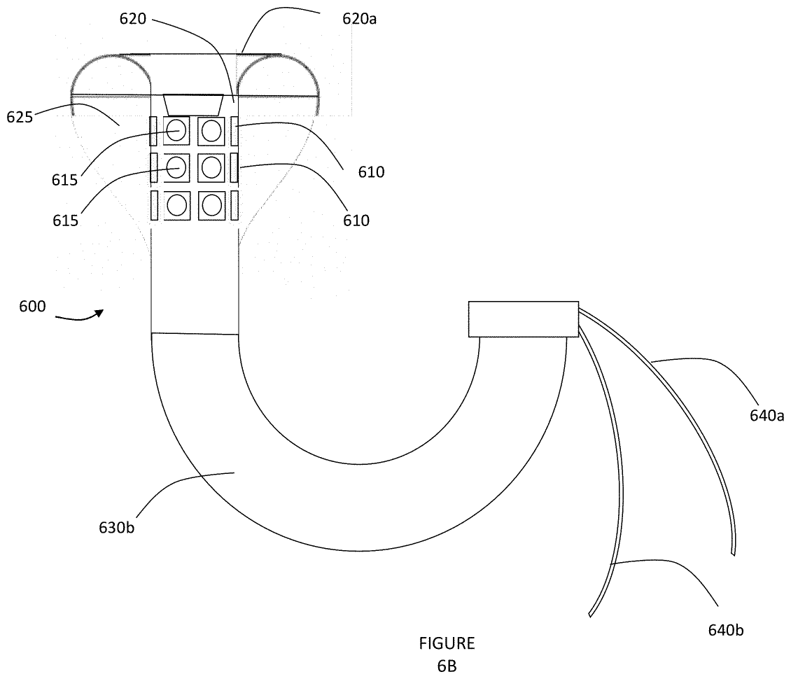

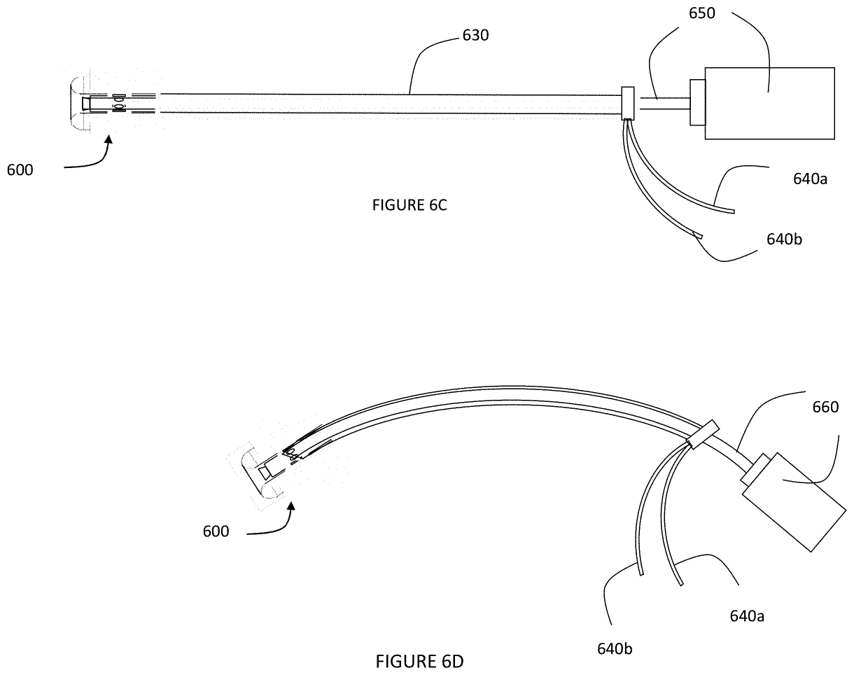

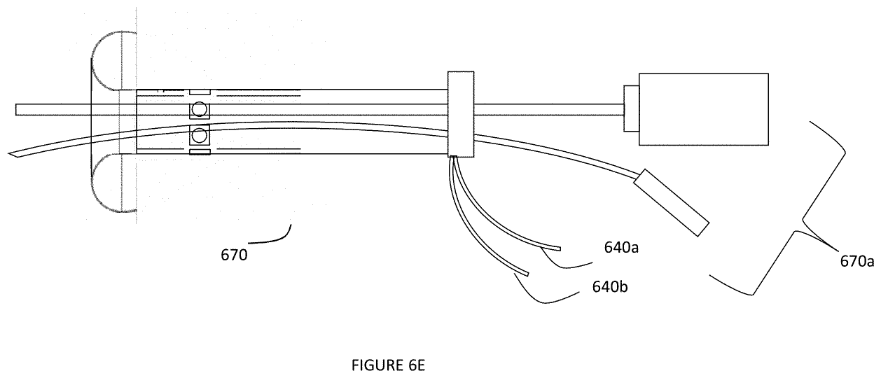

FIGS. 6A to 6E illustrate various embodiments illustrating that a hollow surgical port, flexible or articulating catheter equipped with a separate or built in deployable balloon illuminator 6). In the configuration shown in FIG. 6A, the deployable balloon 625 is a component of a DBI 600 the area in front of surgical port 630a, or catheter distal tip portion 600 which may be used in association with an independent rigid endoscope (shown as element 650 in FIG. 6C), flexible endoscope (shown as element 660 in FIG. 6D), or an articulating stylet equipped with a camera at its distal tip. In the embodiment shown in FIG. 6B, a flexible deployable balloon illuminating catheter 630b may be used FIG. 6C illustrates that a separate rigid endoscope 650 which includes no illumination may be used in association with a separate deployable balloon illuminator 600, where the separately deployable balloon illuminator 600 may provide illumination from illumination sources disposed therein. FIG. 6C illustrates a separate, flexible, or articulating endoscope 660, with or without its own illumination means, can be inserted through a surgical port 630a or a flexible surgical catheter 630b, where inside the body cavity the DBI equipped port or catheter is illuminating the imaging FOV of the separately inserted endoscope at an angle. In each of these configuration, electrical connections 640a may be provided for the illumination sources 610 and 615 and an inlet 640b may be provided for inflation, deflation, and selective deployment control of the DBI 600, which could include circulating air, liquid or get reservoir or bladder connected to the DBI at the distal end, through dual in and out tubes, for circulating the material into the DBI and selective deployment of the DBI

In an alternate embodiment of the independent balloon illuminator shown in FIG. 6E, a distal tip pluggable optoelectronics can be plugged into the DBI tubular body at the distal tip, where other sensors and lighting sources are housed within hermetically sealed pluggable bodies that can separately sterilized and reused. The DBI 600 can be distally built into or inserted over a Single Port 670 used in Single Incision Laparoscopic Surgery (SILS) procedure, where the illumination light from the DBI 600 provides light for the variety of instruments, endoscopes and tools 670a inserted in the single SILS port 670 similar to the DBI 600 in FIG. 6A Examples of endoscopes, ports, catheters, and surgical tools which may comprise separate components which may be used in association with the separately deployable balloon illuminator of this invention are found in at least U.S. Pat. Nos. 8,480,566 and 9,033,870.

As such, in this configuration, the deployable balloon illuminating surgical port or catheter 600 may be a separate product that may be used in association with other existing products so as to improve the operation and use of existing endoscope products 650 that could have their own illumination system or designed without an illumination system (dark scope). In the embodiment shown in FIG. 6A, the separately deployable balloon illuminator 600 includes a combination of LEDs 615 and VCSELs 610 of various wavelengths or colors, turned on and off together, separately or in a specific sequence, where all their lights are processed and mixed inside the DBI 600 for illumination of scenes in front of the DBI 600.

Similar to the configurations described with respect to FIGS. 5A and 5B, the combination of various wavelength LEDs 615 and VSCELs 610 result in a deployable balloon illuminator 600 which is capable of illuminating both visible light and fluorescent light which is subsequently captured by the camera system of an independent or third party endoscope 650. As such, in some embodiments described herein, the deployable balloon illuminator 600 is designed as a separate product which may be used in association with other existing endoscopy products.

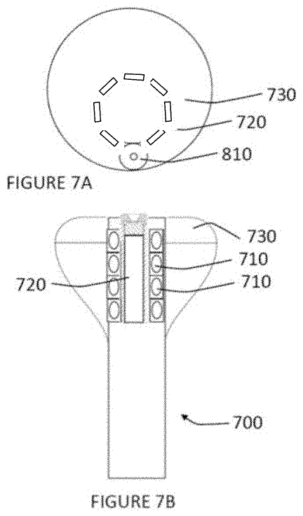

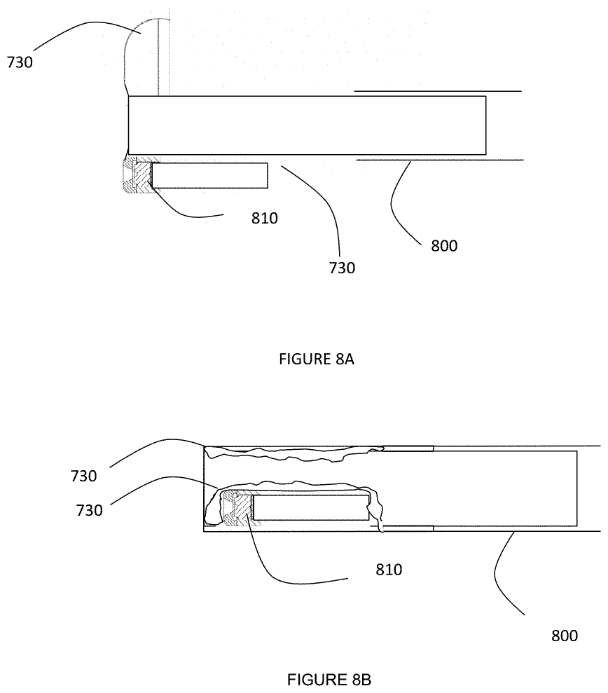

FIGS. 7A-7B and 8A-8B illustrate yet another configuration of a deployable balloon illumination system 700. In this embodiment, the deployable balloon illumination system 700 includes a port or catheter distal tip section 720, and a distal camera 810 is physically attached to its flexible inflatable balloon portion 730 formed on a side thereof, through which the camera 810 may also be deployed. The distal camera 810 is disposed outside of the port or catheter profile 800 during the image capturing process as the deployable balloon 730 is inflated. More specifically, as is shown in FIG. 8B, the camera 810 is configured to be disposed within the port or catheter profile 800 during the camera equipped port or catheter insertion process or while the catheter or port is being placed, while the balloon 730 is in its deflated position. When the camera 810 is deployed (as is shown in FIG. 8A with inflation of the balloon 730), the camera 810 is disposed outside the profile 800 of the port or catheter, so as to free up the port or catheter and provide maximum access internal diameter for insertion of other tools or endoscope. In this configuration, the deployable balloon is both in front of the solid state light sources 710, as well as on the outer side of the camera 801, so it pulls on and deploys the camera 810 outside the port or catheter 720 profile, as the deployable balloon 730 is inflated, mostly in the opposite side of the camera acting as an asymmetrical illumination system 700 for extraction, conditioning and transmission of light from 710 light sources to the front FOV of the camera 810.

Furthermore, in this configuration, a plurality of illumination sources 710 are disposed in form of an array outside the sides of the port or catheter distal tip 720 where their electrical power connections as well the camera 810 electrical lines could run within a separate lumen in the body or wall of the port or catheter 720. As was previously described, the illumination sources 710 may include a variety of different types of illumination sources, including any desired combination of LEDs. VCSELs, or other light sources which may provide the desired type of illumination, of sequence of various illumination for the particular application and use of the endoscope. The 810 camera equipped port or catheter 720 could be used as an auxiliary camera with a broader FOV of the general surgical site, in conjunction with the main endoscope or a camera stylet, used separately inside the port or catheter 720, where the illumination sources 710 could provide illumination for both camera 810 as well as the separate dark or illuminated endoscope or camera equipped articulating stylet used inside the port or catheter 720.

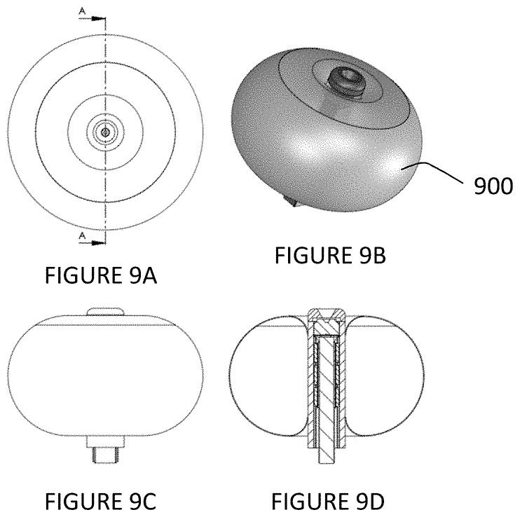

Although the deployable balloon endoscope systems described above with respect to FIGS. 1 to 8B each have a heart or bulb shape, the invention is not so limited and any number of different shaped deployable balloon shapes may be used so as to increase the efficiency at the site to be imaged by the deployable balloon illuminated endoscope. One example of an alternative shape which may be used is the apple shaped deployable balloon endoscope 900 shown in FIGS. 9A-9D. It should be understood that a variety of other shaped balloon shapes, sizes, and configurations may be used, not only to provide other functionalities to the device, such as providing space in front of the endoscope where there is limited space available in front and around the distal tip for an appreciable working distance, to fill in a gap within a body organ such as plugging a natural orifice while operating through a working channel of the DBI endoscope as described above, variety of thin film coatings can be used on the DBI, filtering or reflecting various wavelengths of light, or surface features molded in variety of locations and surfaces of the deployable balloon illuminator, for optical and non-optical purposes without departing from the scope of the application.

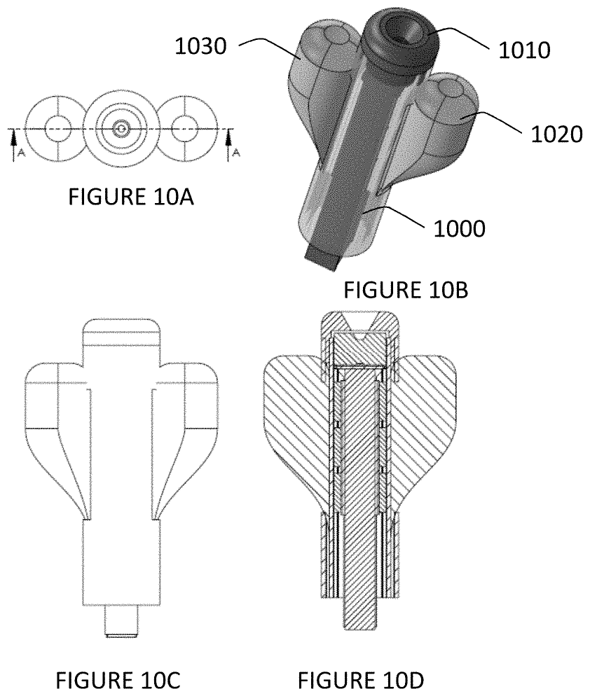

FIGS. 10A-10D show yet another embodiment of a deployable balloon illuminating endoscope 1000, wherein two separately deployable balloon portions 1020 and 1030 are shown and are disposed on either side of a camera system 1010. As may be understood, the two separate deployable portions 1020 may be independently deployed and/or illuminated according to the specific needs of the application and it should be understood that any number of independently controlled deployable balloon illuminators may be used in association with the camera and imaging system described herein without departing from the scope of the invention. Variety of deployable balloon illuminators can be also used along the body of the device as well as the distal tip of the device. The deployable balloon illuminators could also be used, when deployed together or alternately such as the two deployable illuminators 1030 and 1020 on either side of the device, to articulate, position, bend and turn the flexible distal tip itself, inserted in the body orifice or cavity, as each of the deployable 1030 or 1020 are alternately deployed, bending and turning the device distal tip portion.

As may be understood by one of skill in the art, one advantage of the embodiments described herein is that the systems described herein enable the placement of illumination light towards an object inside the body in diagnostic or surgical procedures, while providing an illuminated endoscope system which has little loss in conjunction with the transmission of light from the external source to the surgical site. Furthermore, the embodiments are able to provide a large array of light transmission sources which can also include different types of light transmission sources without enlarging the size of the distal end of the endoscope. This provides an endoscope which has a small cross-sectional profile, making it easier to travel through small passages in the human body. Once the endoscope has been placed at a location which a physician or other health care provider wishes to image, the embodiments herein also provide a safely and easily deployed balloon which enlarges the cavity to be imaged and also acts as a reflective illuminator which assists in lighting the cavity. As such, the deployable balloons described herein also assist in positioning the imaging system and transmitting light to the desired area of the cavity, allowing higher power solid state light sources to be used distally with well managed and safe heat dissipation mechanism through the DBI itself, where the light extraction efficacy as well as pointing and uniformity of the illumination light are improved by the DBI.

The embodiments described herein are able to be used in association with LEDs or other light sources which have equal efficiency in converting electrical power to useful light, can be operated in much lower input power, eliminating the need for sophisticated power and heat management. Power and control signals transmitting through appropriate wires and flex circuitry, can be easily routed along the rigid, flexible, partially flexible, or articulating tool or endoscope body to the illumination sources.

Although not expressly described herein, miniature, optical components such as lenses, miniature autofocus mechanisms, mirrors, beam splitters, polarizers, waveplates, etc. can also be used in conjunction with the light emitting sources, camera, or other optical components, as well as the DBI itself and optical features built in, molded within, or thin film coatings deposited on its surfaces, is used to further manipulate the illumination characteristics of the light or the ability to capture the reflected light by the imaging optics of a DBI incorporated endoscope or a separate endoscope. The DBI surface curvature, optional surface features, and filling material could be designed to act as a lens for example, to direct the light to larger or smaller areas of the scene, or focusing the beam to a small area on the object depending on the application.

Polarization and wavelength characteristics of the solid state laser or LED light output can also be used in special detection schemes, where depth perception or other biological imaging characteristics that depend on the polarization or wavelength of the light can be better perceived, similar to polarized microscopy or through IR tissue penetrated imaging to locate veins, etc. Miniature Deployable Balloon illuminators can be used on access devices such as speculum distal tips to look into nose, ear, oral cavity, vagina or other natural orifices. Deployable Balloon illuminators can also be used in catheters that enter and travel through orifices and lumens filled with urine or blood, where specialized cameras need specific illumination and excitation sources, with narrow band lasers or wide band LED sources, in infrared, spectral or speckle imaging.

The present invention may be embodied in other specific forms without departing from its spirit or essential characteristics. The described embodiments are to be considered in all respects only as illustrative and not restrictive. The scope of the invention is, therefore indicated by the appended claims rather than by the foregoing description. All changes which come within the meaning and range of equivalency of the claims are to be embraced within their scope.

1. A selectively deployable balloon member for insertion into and illumination of a body cavity, the selectively deployable balloon member comprising:
a flexible tubular body for insertion into the body cavity having a non-deployed configuration during insertion into the body cavity and a deployed configuration after insertion into the body cavity, the flexible tubular body being inflated after the insertion into the body cavity so as to:
receive light from at least one light source, and
selectively transmit that light out through at least one optically transparent portion of the flexible tubular body to an object of interest in the body cavity in the deployed configuration through the inflation of the flexible tubular body.

2. The selectively deployable balloon member of claim 1, wherein a portion of the flexible tubular body is expandable in volume and is configured to be filled with air, gel, or fluid or combination thereof when in the deployed configuration.

3. The selectively deployable balloon member of claim 2, wherein the flexible tubular body is inflated to fill a gap between the flexible tubular body and the at least one light source with material possessing higher refractive index than air so as to cause more effective extraction of light from the at least one light source as the flexible tubular body is inflated.

4. The selectively deployable balloon member of claim 1, wherein a surface of the flexible tubular body, optical features molded within, optical coatings deposited on it, or optical properties of a material filled therein in the deployed configuration are configured to condition light received from the at least one light source by means of selective TIR, reflection, refraction, wavelength and or polarization filtration, scattering, diffracting, focusing, and/or dispersion.

5. The selectively deployable balloon member of claim 1, wherein the flexible tubular body has a symmetrical heart, apple, general bulb shape, asymmetrical, or sectional expanding shape in the deployed configuration.

6. The selectively deployable balloon member of claim 5, wherein a shape of the flexible tubular body and any pre-molded optical features on a surface of the flexible tubular body or within the flexible tubular body, optically, sectionally and spatially condition illumination light emitted from at the least one light source so as to modify a spatial or directional distribution of the light as it passes through the flexible tubular body, causing the light to be selectively redistributed as it passes through an interior the flexible tubular body and is selectively reflected from interior walls of flexible tubular body and exits the flexible tubular body through the at least one optically transparent portion of the flexible tubular body.

7. The selectively deployable balloon member of claim 1, the flexible tubular body being deployed via at least one tube or lumen housed within the flexible tubular body which inflates the flexible tubular body through an operation of a plurality of valves, and possible intermediate reservoirs and bladders, incorporated inside or proximally outside the flexible tubular body.

8. The selectively deployable balloon member of claim 7, wherein the plurality of valves controls selective deployment of the flexible tubular body in parts or as a whole, through a single or circular tubing for the selective inflastion of deployable balloon member or members.

9. The selectively deployable balloon member of claim 1, wherein the flexible tubular body has a toroid shape with a recess formed in a distal end therein which houses a camera of a medical scope or otherwise acts as a stopping and positioning for an independently inserted endoscope.

10. The selectively deployable balloon member of claim 1, wherein the at least one light source emits light laterally from an interior of the selectively deployable balloon member to the flexible tubular body, the light being reflected from a reflective portion toward the optically transparent portion of the flexible tubular body.

11. The selectively deployable balloon member of claim 1, wherein the at least one light source comprises a solid state light source positioned on a back side of the flexible tubular body during insertion into the body cavity and which is deployed outward from a lateral facing direction when the flexible tubular body is inflated.

12. The selectively deployable balloon member of claim 1, wherein light transmitted from the at least one optically transparent portion exiting the flexible tubular body is conditioned to act as a spotlight for illumination of an endoscope FOV (Field Of View), and moved with an articulating endoscope to have the illumination and endcoscope FOV collinear and overlapping.

13. The selectively deployable balloon member of claim 1, wherein the at least one light source is a light emitting diode or a vertical cavity surface emitting laser (VCSEL) of various wavelengths.

14. The selectively deployable balloon member of claim 13, wherein an optical geometry, surface properties, material composition of the flexible tubular body, optical coatings deposited on the flexible tubular body, or a material the flexible tubular body is filled when being inflated condition and spatially mix wavelengths of light received from the at least one light source.

15. The selectively deployable balloon member of claim 14, wherein a timing when at least a portion of the flexible tubular body is inflated is sequentially synchronized with turning on or off of the at least one light source.

16. A device for insertion into and illumination of a body cavity, the device comprising:
a selectively deployable balloon member having a non-deployed configuration during insertion into the body cavity and a deployed configuration after insertion into the body cavity, the selectively deployable balloon member being inflated after the insertion into the body cavity so as to be in the deployed configuration and having:
at least one reflective portion configured to reflect illuminated light received from at least one light source disposed on an interior of the selectively deployable balloon member when in the deployed configuration, and
at least one transparent portion located at a distal end thereof which enables illuminated light from the at least one light source to pass therethrough.

17. The device of claim 16, wherein the selectively deployable balloon is configured to be filled fully or partially with air, gel, or fluid, or combination thereof, when in a fully or partially deployed configuration.

18. The device of claim 16, wherein the selectively deployable balloon or portions thereof have a symmetric heart, apple, bulb shape, asymmetrical, or sectional expanding shape, when in a partially or fully deployed configuration.

19. The device of claim 16, the selectively deployable balloon being deployed via at least one tube or lumen which inflates an interior of the selectively deployable balloon as exterior walls thereof expand.

20. The device of claim 16, the selectively deployable balloon having a rigid recess member or window formed therein for positioning a camera of a medical scope, at the distal end of a hollow section to house the separate camera of the medical scope.

21. The device of claim 16, wherein the at least one light source emits light at an angle from the interior of the selectively deployable balloon toward an exterior of the selectively deployable balloon, the light being reflected from the reflective portion toward the transparent portion of the selectively deployable balloon.

22. The device of claim 16, wherein the light source is a light emitting diode or a vertical cavity surface emitting laser (VCSEL) of various wavelengths, which is selectively positioned, selectively, or concurrently turned on with respect to an inflation of the selectively deployable balloon member.

23. An illumination device for insertion into a body cavity, the illumination device comprising:
an elongate hollow tube with an internal lumen between a distal opening at a distal end and a proximal opening at a proximal end, the elongate hollow tube including a rigid distal portion associated with the distal opening configured to be at least partially inserted into the body cavity;
a selectively deployable balloon member operably coupled to the rigid distal portion and having an insertion position and a deployed position, the selectively deployable balloon member having:
an interior portion configured to receive light from at least one electro-optic illumination element coupled to the selectively deployable balloon member, wherein the at least one electro-optic illumination element provides illumination at one or more electromagnetic wavelengths and is configured to be selectively turned on or off;
a reflective portion configured to reflect illuminated light;
a transparent portion located at a distal end thereof which enables illuminated light to pass therethrough; and
an imaging element housing portion positioned in the rigid distal portion which is configured to house an imaging element when coupled thereto.

24. The illumination device of claim 23, wherein the at least one electro-optic illumination element is disposed within an interior of the selectively deployable balloon member and is configured such that when the at least one electro-optic illumination element is turned on, light from the at least one electro-optic illumination element is reflected from the reflective portion of the selectively deployable balloon member and is transmitted through the transparent portion of the selectively deployable balloon member towards the rigid distal portion where the imaging element is disposed.

25. The illumination device of claim 23, wherein the selectively deployable balloon member is configured to be filled with air, gel, fluid, or combination thereof when in a deployed configuration.

26. The illumination device of claim 23, wherein the selectively deployable balloon member has a symmetric heart, apple, bulb shape, asymmetrical, or sectional expanding shape when in a deployed configuration.

27. The illumination device of claim 23, wherein the imaging element housing provides the imaging element a recessed positional alignment once the imaging element is positioned inside the deployable balloon member.

28. The illumination device of claim 27, wherein the imaging element is permanently built in, or otherwise inserted through the elongate hollow tube, via a separate endoscope body, positioned inside the imaging element housing portion of the deployable balloon member.

29. The illumination device of claim 28, wherein the separate endoscope body has light sources positioned on an outer wall thereof which couple with a transparent inner wall of the hollow elongate tube or transparent windows within the hollow elongate tube, as the separate endoscope is distally positioned within the imaging element housing portion of the selectively deployable balloon.

30. The illumination device of claim 23, wherein the interior portion comprises a transparent window, with or without optical filteration properties (in wavelength or polarization) portion which is disposed to receive light from at least one electro-optic illumination element.

31. The illumination device of claim 30, where in another body inserted into the elongate hollow tube comprises illumination sources disposed in front of a transparent inner wall of the hollow elongate tube.

32. The illumination device of claim 23, wherein the at least one electro-optic illumination element emits light laterally towards an interior of the selectively deployable balloon, the light being reflected from the reflective portion toward the transparent portion of the selectively deployable balloon.

33. An illumination device for insertion into a body cavity, the illumination device comprising:
an elongate hollow tube with an internal lumen between a distal opening at a distal end and a proximal opening at a proximal end, the elongate hollow tube including a rigid distal portion associated with the distal opening configured to be at least partially inserted into the body cavity;
a selectively deployable balloon member operably coupled to the rigid distal portion and having an insertion position and a deployed position, the selectively deployable balloon member having:
an interior portion configured to receive light from at least one electro-optic illumination element coupled to the selectively deployable balloon member, wherein the at least one electro-optic illumination element provides illumination at one or more electromagnetic wavelengths and is configured to be selectively turned on or off;
a reflective portion configured to reflect illuminated light;
a transparent portion located at a distal end thereof which enables illuminated light to pass therethrough; and
where an imaging element is interiorly attached to the selectively deployable balloon member and is positioned within the hollow elongate tube in the insertion position and is deployed outward by deploying to an operational position outside a profile of the hollow elongate tube with an inflation of the selectively deployable balloon member to the deployed position.
